(19) United States
(12) Patent Application Publication
(10) Pub. No.: US 2025/0073043 A1
(43) Pub. Date: Mar. 6, 2025

(54) INTERNAL FIXATION DEVICE FOR FUSION AND RECONSTRUCTION OF SPINOUS PROCESSES AND VERTEBRAL LAMINAE

(71) Applicant: Shenzhen YuanShengLin Technology Company Limited, Shenzhen (CN)

(72) Inventor: Baoming Qiu, Shenzhen (CN)

(21) Appl. No.: 18/820,217

(22) Filed: Aug. 29, 2024

(30) Foreign Application Priority Data
Aug. 30, 2023 (CN) 202311112801.2

(51) Int. Cl. A61F 2/44; A61F 2/30

(52) U.S. Cl. CPC A61F 2/4405; A61F 2/30771; A61F 2002/30405; A61F 2002/30772; A61F 2002/3092; A61F 2002/3093

(57) ABSTRACT

An internal fixation device for fusion and reconstruction of spinous processes and vertebral laminae, which includes a bone fusion structure and a rigid skeleton body, the rigid skeleton body includes a first rigid plate and a second rigid plate, the bone fusion structure is located between two rigid plates. The bone fusion structure is a microporous structure with a storage channel inside for storing bone growth promoting substances, front and back sides of the bone fusion structure have a medicine inlet and a medicine outlet, the medicine outlet is docked with anterior and posterior spinous processes, facilitating bone fusion of a cross-section position of the spinous processes. The two rigid plates are extended outward and downward to form the lamina fixation part, covering and reconstructing the vertebral laminae, and the two rigid plates are fixedly connected to the anterior and posterior spinous processes and the vertebral laminae by screws.

### CROSS-REFERENCE TO RELATED APPLICATIONS

The present application claims priority of Chinese Patent Application No. 202311112801.2, filed on Aug. 30, 2023, the entire contents of which are hereby incorporated by reference.

### TECHNICAL FIELD

The present disclosure relates to a field of medical devices, and in particular to an internal fixation device for fusion and reconstruction of spinous processes and vertebral laminae.

### BACKGROUND

Orthopedic surgery, especially spinal surgery, requires internal fixation in addition to decompressing the neural spinal canal. Currently, the internal fixation usually uses pedicle screws or intervertebral bone fusion structures. However, using the pedicle screws has problems such as large exposure range, large soft tissue injury, and complications caused by screws compressing nerves and blood vessels. The intervertebral bone fusion structures, whether it is PLIF, TLIF from the back, OLIF, XLIF from the side, or ALIF from the front, exposed paths are all relatively complex, and intervertebral discs are difficult to handle clean, if bony vertebral laminae are fractured, there is a risk of collapse of the bone fusion structures and even damage to nerves and blood vessels. In order to decompress, a spinous process and vertebral lamina structure at the back is removed, nerve scar adhesion is easy to be caused in the future, and the nerves also lose protection from the back. If internal fixation can be performed on spinous process bones and the vertebral laminae at a superficial position at the back, at the same time, the removed spinous process and vertebral lamina structure is fused and reconstructed, the exposure range is small, an operation is easier, and a surgical risk is lower.

### SUMMARY

A purpose of the present disclosure is to provide an internal fixation device for fusion and reconstruction of spinous processes and vertebral laminae, which can fuse spinous process bones and the vertebral laminae at a superficial position at the back of spines, with small exposure range, easy operation and low surgical risk.

The present disclosure is achieved as follows, an internal fixation device for fusion and reconstruction of spinous processes and vertebral laminae, including a rigid skeleton body and a bone fusion structure, the rigid skeleton body includes a first rigid plate and a second rigid plate, the bone fusion structure is located between the first rigid plate and a second rigid plate, and the first rigid plate and the second rigid plate are located on a left side and a right side of the bone fusion structure respectively; a microporous structure is provided inside the bone fusion structure, and an orifice of the microporous structure is located on a side surface of the bone fusion structure;

both the first rigid plate and the second rigid plate includes a bone fusion structure fixed part and a vertebral lamina fixed part, the bone fusion structure fixed part is fixedly connected to the bone fusion structure, and a top of the vertebral laminae fixed part is fixedly connected to a bottom of the bone fusion structure fixed part; and

the first rigid plate includes an anterior spinous process fixed part and a posterior spinous process fixed part, the anterior spinous process fixed part and the posterior spinous process fixed part are fixedly connected to a front end and a back end of the bone fusion structure fixed part respectively, and protruded from a front end surface and a back end surface of the bone fusion structure respectively; the anterior spinous process fixed part is provided with a first screw hole, and is connected to an anterior spinous process and an anterior vertebral lamina by a first screw; and the posterior spinous process fixed part is provided with a second screw hole, and is connected to a posterior spinous process and a posterior vertebral lamina by a second screw.

Compared with the prior art, the present disclosure includes the following beneficial effects:

The present disclosure provides the internal fixation device for fusion and reconstruction of spinous processes and vertebral laminae, which includes the bone fusion structure and the rigid skeleton body, and the bone fusion structure is embedded and fixed in the rigid skeleton body; a storage channel used to store bone fusion-promoting substances is located inside the bone fusion structure, a top surface of the bone fusion structure is provided with a medicine inlet, and both a front end face and a back end face of the bone fusion structure are provided with a medicine outlet; and the medicine inlet located on the top surface allows doctors to easily put in medicine that promotes bone formation, and the medicine outlet are connected to anterior and posterior spinous processes, which facilitates the spinous processes to absorb the medicine and is conducive to the rehabilitation of a cross-section position of the spinous processes. The vertebral lamina fixed part is formed by the first rigid plate and the second rigid plate being extended downward and into an outward direction, to cover and reconstruct the vertebral laminae, the first rigid plate and the second rigid plate are connected and fixed to the anterior and posterior spinous processes and the vertebral laminae by screws. The entire internal fixation device is located on the vertebral lamina at the back of the spine and between the anterior and posterior spinous processes, with a small exposure range, easy operation and low surgical risk; moreover, the internal fixation device can prevent the device from collapsing and avoid nerve injury by the vertebral laminae fixed parts, therefore, the internal fixation device is a new type of internal fixation device which can reconstruct and fuse the spinous processes and the vertebral laminae.

### BRIEF DESCRIPTION OF DRAWINGS

FIG. 1 is a structural schematic diagram of an internal fixation device for fusion and reconstruction of spinous processes and vertebral laminae according to Embodiment 1 of the present disclosure;

FIG. 2 is a structural schematic diagram of the internal fixation device for fusion and reconstruction of spinous processes and vertebral laminae shown in FIG. 1 from another perspective;

FIG. 3 is a top view schematic diagram of the spinous process and lamina fixed by the internal fixation device for fusion and reconstruction of spinous processes and vertebral laminae according to Embodiment 1 of the present disclosure;

FIG. 4 is a left view schematic diagram of the spinous process lamina fixed by the internal fixation device for fusion and reconstruction of spinous processes and vertebral laminae according to Embodiment 1 of the present disclosure;

FIG. 5 is a structural schematic diagram of the internal fixation device for fusion and reconstruction of spinous processes and vertebral laminae using a large hole design for a medicine inlet and a medicine outlet according to Embodiment 1 of the present disclosure;

FIG. 6 is a schematic diagram of the internal fixation device for fusion and reconstruction of spinous processes and vertebral laminae using a split structure design for a bone fusion structure and a rigid skeleton body according to Embodiment 1 of the present disclosure;

FIG. 7 is a structural schematic diagram of an internal fixation device for fusion and reconstruction of spinous processes and vertebral laminae according to Embodiment 2 of the present disclosure; and

FIG. 8 is a schematic diagram of the internal fixation device for fusion and reconstruction of spinous processes and vertebral laminae using a split structure design for a first rigid plate and a second rigid plate according to Embodiment 3 of the present disclosure.

### REFERENCE NUMERALS IN THE DRAWINGS

1, bone fusion structure; 11, medicine storage channel; 12, medicine inlet; 13, medicine outlet; 14, microporous structure; 2, rigid skeleton body; 21, first rigid plate; 211, first bone fusion structure fixed part; 212, first vertebral lamina fixed part, 213, anterior spinous process fixed part; 214, posterior spinous process fixed part; 22, second rigid plate; 221, second bone fusion structure fixed part; 222, second vertebral lamina fixed part, 23, intermediate connecting plate, 24, screw hole; 3, screw;

100, anterior spinous process; 200, posterior spinous process; 300, anterior vertebral lamina; 400, posterior vertebral lamina.

### DESCRIPTION OF EMBODIMENTS

In order to clarify the purpose, technical solutions and beneficial effects of the present disclosure, the present disclosure is further described in detail below with reference to accompanying drawings and embodiments. It should be understood that specific embodiments described herein are used to illustrate the present disclosure, but not used to limit the present disclosure.

In the description of the present disclosure, it should be noted that the orientation or positional relationship indicated by the terms “center”, “up”, “down”, “left”, “right”, “front”, “back”, “vertical”, “horizontal”, “inside”, “outside” and the like is based on the orientation or positional relationship shown in the accompanying drawings, which is only for the convenience of describing the present disclosure and simplifying the description, rather than indicating or suggesting that a device or a component referred to must have a specific orientation and be constructed and operated in a specific orientation, and therefore the terms cannot be understood as limiting the present invention. The terms “first”, “second” and “third” are just used for descriptive purposes, and cannot be understood as indicating or implying relative importance. In addition, unless otherwise expressly specified or limited, the terms “install” ″, “connected” and “communication” should be understood in a broad sense, for example, it can be fixed connection, removable connection, or integrated connection; it can be direct connection, indirect connection through an intermediate medium, or it can also be internal connection between two components. For those skilled in the art, specific meanings of the above terms in the present disclosure can be understood according to specific circumstances.

### Embodiment 1

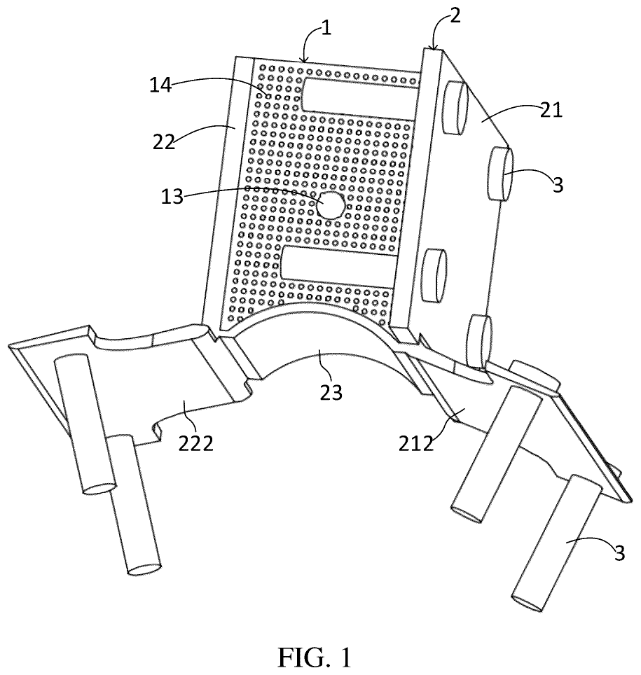

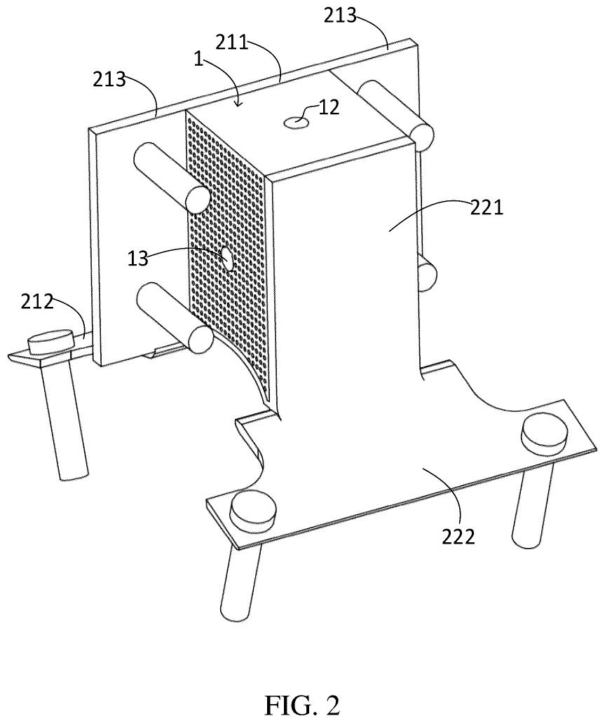

Referring to FIG. 1 and FIG. 2, an internal fixation device for fusion and reconstruction of spinous processes and vertebral laminae provided in the embodiment 1 is shown, including a bone fusion structure 1 and a rigid skeleton body 2. The rigid skeleton body 2 includes a first rigid plate 21, a second rigid plate 22 and an intermediate connecting plate 23.

The intermediate connecting plate 23 is fixedly connected between the first rigid plate 21 and the second rigid plate 22, and the bone fusion structure 1 is located on the intermediate connecting plate 23. The first rigid plate 21 and the second rigid plate 22 are respectively located on a left side and a right side of the bone fusion structure 1, in the embodiment, the first rigid plate 21, the second rigid plate 22 and the intermediate connecting plate 23 are an integrated structure.

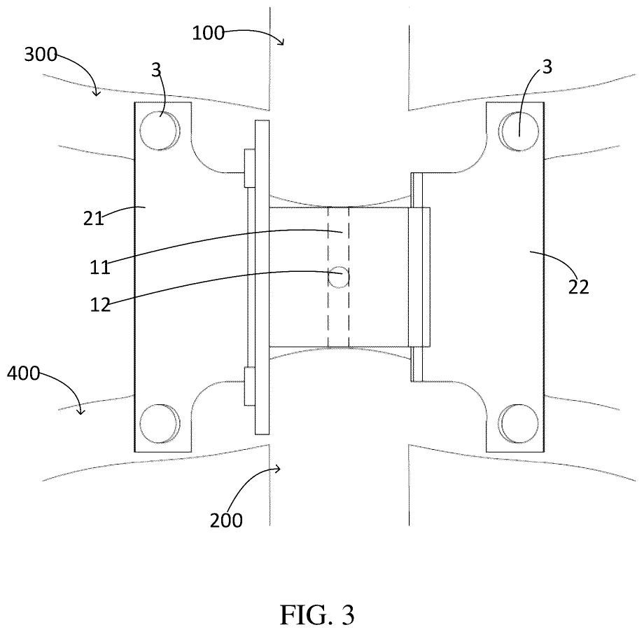

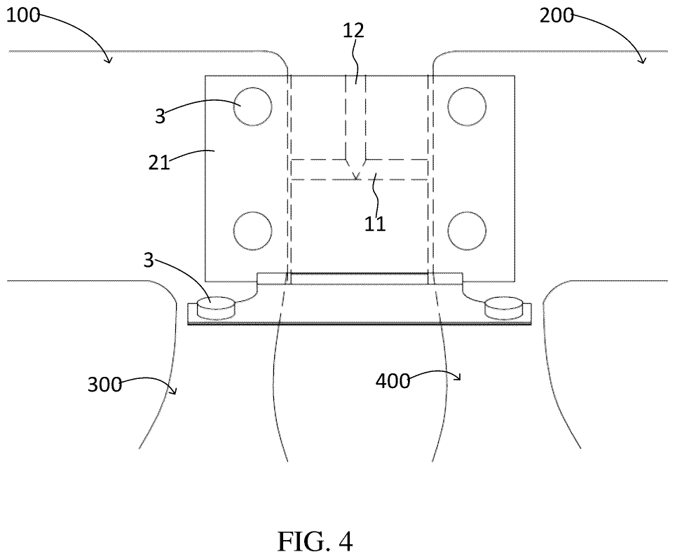

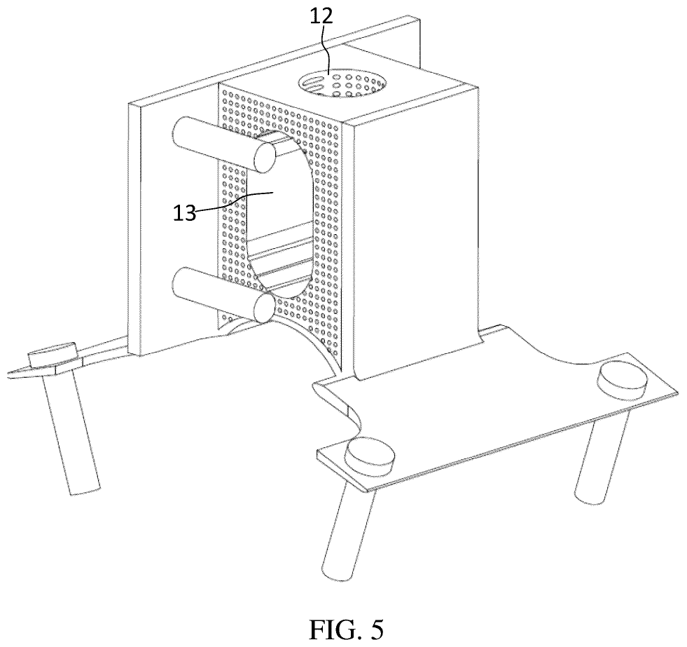

A medicine storage channel 11 is located inside the bone fusion structure 1, a top surface of the bone fusion structure 1 is provided with a medicine inlet 12, both a front end face and a back end face of the bone fusion structure 1 are provided with a medicine outlet 13, and both the medicine inlet 12 and the medicine outlet 13 are in communication with the medicine storage channel 11. The medicine inlet 12 located on the top surface allows doctors to easily put in medicine that promotes bone formation, referring to FIG. 3 and FIG. 4 together, the medicine outlet 13 located on the front end face and the medicine outlet 13 located on the back end face are connected to an anterior spinous process 100 and a posterior spinous process 200 respectively, which facilitates spinous processes to absorb the medicine and is conducive to the rehabilitation of a cross-section position of the spinous processes. In practical applications, when a large amount of medicine needs to be put in, in order to increase an amount and a speed of medicine input, the medicine inlet 12 and the medicine outlet 13 can be designed as a large hole type. Referring to FIG. 5, a size of the medicine inlet 12 accounts for ⅓ to ½ of a top surface area of the bone fusion structure 1, and a size of the medicine outlet 13 accounts for ⅓ to ½ of a side surface area of the bone fusion structure 1.

Specifically, the first rigid plate 21 includes a first bone fusion structure fixed part 211, a first vertebral lamina fixed part 212, an anterior spinous process fixed part 213, and a posterior spinous process fixed part 214.

The first bone fusion structure fixed part 211 is fixedly connected to a left side surface of the bone fusion structure 1, and the top of the first vertebral lamina fixed part 212 is fixedly connected to the bottom of the first bone fusion structure fixed part 211. In the embodiment, the first vertebral lamina fixed part 212 is a bending structure relative to the first bone fusion structure fixed part 211, and the first vertebral lamina fixed part 212 is formed by the bottom of the first bone fusion structure fixed part 211 being inclined extended downward and into an outward direction. One or more screw holes are provided at both front and back ends of the first vertebral lamina fixed part 212, and are connected to an anterior vertebral lamina 300 and a posterior vertebral lamina 400 on the left side by one or more screws 3 respectively.

The anterior spinous process fixed part 213 and the posterior spinous process fixed part 214 are fixedly connected to front and back ends of the first bone fusion structure fixed part 211 respectively, and are protruded from both front and back end surfaces of the bone fusion structure 1. One or more screw holes are provided on the anterior spinous process fixed part 213, and are connected to the anterior spinous process 100 by one or more screws 3; and one or more screw holes are provided on the posterior spinous process fixed part 214, and are connected to the posterior spinous process 200 by one or more screws 3.

The second rigid plate 22 includes a second bone fusion structure fixed part 221 and a second vertebral lamina fixed part 222. The second bone fusion structure fixed part 221 is fixedly connected to a right side surface of the bone fusion structure 1, and the top of the second vertebral lamina fixed part 222 is fixedly connected to the bottom of the second bone fusion structure fixed part 221. In the embodiment, the second vertebral lamina fixed part 222 is a bending structure relative to the second bone fusion structure fixed part 221, and the second vertebral lamina fixed part 222 is formed by the bottom of the second bone fusion structure fixed part 221 being inclined extended downward and into an outward direction. Both front and back ends of the second vertebral lamina fixed part 222 are provided with one or more screw holes, and are connected to an anterior vertebral lamina 300 and a posterior vertebral lamina 400 on the right side by one or more screws 3 respectively.

In addition, the first vertebral lamina fixed part 212 and the second vertebral lamina fixed part 222 can also be fixed to the anterior vertebral lamina 300/the posterior vertebral lamina 400 and an articular process by one or more screws 3; or the first vertebral lamina fixed part 212 and the second vertebral lamina fixed part 222 can also be fixed to the articular process and a pedicle of vertebral arch by one or more screws 3.

Further, a bottom shape of the bone fusion structure 1 is matched with a top shape of the intermediate connecting plate 23, and a bottom shape of the intermediate connecting plate 23 is matched with a top shape of the vertebral lamina, so that a bottom surface of the intermediate connecting plate 23 fits a top surface of the vertebral lamina. In the embodiment, the bottom of the bone fusion structure 1 and the intermediate connecting plate 23 are both arc-shaped structures.

Since a shape of the intermediate connecting plate 23 is matched with the top shape of the vertebral lamina, and the first vertebral lamina fixed part 212 and the second vertebral lamina fixed part 222 are extended into the outward direction, so that an overall shape of the bottom of the internal fixation device is close to the vertebral lamina, thereby covering spinal canals, reconstructing the spinal canals, and limiting a depth of the internal fixation device.

Both the first rigid plate 21 and the second rigid plate 22 are connected and fixed to the anterior and posterior spinous processes and vertebral laminae by the screws 3. The entire internal fixation device is located on the posterior vertebral lamina of the spine and between the anterior and posterior spinous processes, with a small exposure range, easy operation and low surgical risk. Moreover, the internal fixation device can prevent the device from collapsing and avoid nerve injury by the vertebral laminae fixed parts, therefore, the internal fixation device is a new type of internal fixation device which can reconstruct and fuse the spinous processes and vertebral laminae.

Further, a microporous structure 14 is provided inside the bone fusion structure 1, and orifices of the microporous structure 14 are located on front and back side surfaces of the bone fusion structure 1. In the embodiment, the microporous structure 14 is composed of a plurality of micropores distributed in an array, and a length direction of the plurality of micropores penetrates the front and back end surfaces of the bone fusion structure 1. The microporous structure 14 is conducive to bone tissue to grow into the bone fusion structure 1, thereby achieving a fusion effect of the bone tissue and bone fusion structure 1. It is easy to understand that the rigid skeleton body 2 can also be left in a human body together with the bone fusion structure 1, at this time, contact positions of both the first rigid plate 21 and the second rigid plate 22 of the rigid skeleton body 2 with the human bone tissues can also be provided with the microporous structure 14, to achieve the fusion of the bone tissue and the rigid skeleton body 2.

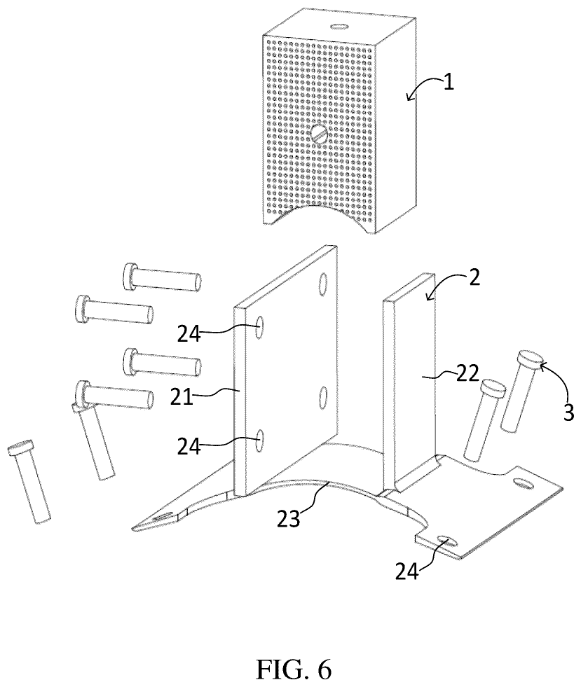

In practical applications, a material of the bone fusion structure 1 can be selected from titanium alloy, 3D printing materials, PEEK (Polyether Ether Ketone) material, artificial bone, allogeneic bone and the like. A material of the rigid skeleton body 2 is a metal material, such as steel, titanium, alloy, titanium alloy and the like. It is easy to understand that when the material of the bone fusion structure 1 is the same as the material of the rigid skeleton body 2, the bone fusion structure 1 and the rigid skeleton body 2 can be an integral structure and can be produced by an integral molding method. When the material of the bone fusion structure 1 is different from the material of the rigid skeleton body 2, the bone fusion structure 1 can be embedded in the rigid skeleton body 2, and then the rigid skeleton body 2 and the bone fusion structure 1 are fixedly connected by small screws (as shown in FIG. 6). A specific shape of the bottom of the bone fusion structure 1 is not limited in the present disclosure, and the bone fusion structure 1 can be produced in a customization mode, to match different incisions or fracture shapes of the spinous processes and vertebral laminae.

Further, one end of the first vertebral lamina fixed part 212 connected to the first bone fusion structure fixed part 211 is a first connecting end, the other end is a first free end, and a width of the first connecting end is smaller than a width of the first free end. One end of the second vertebral lamina fixed part 222 connected to the second bone fusion structure fixed part 221 is a second connecting end, the other end is a second free end, and a width of the second connecting end is smaller than a width of the second free end. By making the connection narrower, the connection can be easily deformed to achieve an effect of an elastic arm, therefore, the first bone fusion structure fixed part 211 can be bent relative to the first vertebral lamina fixed part 212, so that the first vertebral lamina fixed part 212 can better fit with the vertebral laminae on the left side. Similarly, the second bone fusion structure fixed part 221 can be bent relative to the second vertebral lamina fixed part 222, so that the second vertebral lamina fixed part 222 can better fit with the vertebral laminae on the right side.

Further, a width of the second bone fusion structure fixed part 221 of the second rigid plate 22 is equal to a width of the right side surface of the bone fusion structure 1. A width of the first bone fusion structure fixed part 211 of the first rigid plate 21 is equal to a width of the left side surface of the bone fusion structure 1. The bone fusion structure fixed parts and the bone fusion structure 1 are designed to be of equal width, while fully protecting a connection strength between the rigid plates and the bone fusion structure 1, it is also conducive to reducing an area of the rigid plates, saving the cost of the rigid plates, and preventing the rigid plates from being too large to affect adjacent human tissue. In other embodiments, the width of the second bone fusion structure fixed part 221 and the width of the first bone fusion structure fixed part 211 can also be smaller than the width of the side surfaces of the bone fusion structure 1. A size and a specific shape of each of the first rigid plate 21 and the second rigid plate 22 are not limited in the present disclosure.

In order to more clearly illustrate the technical effects of the internal fixation device of the embodiment, some specific application scenarios are given as examples:

Practical application example 1: during posterior spinal surgery, a supraspinous ligament and an interspinous ligament in interspinous processes are removed, parts of the superior and inferior spinous processes and vertebral laminae are treated, a spinous process bone graft surface is created, a yellow ligament is removed, a lateral recess nerve root canal is released, and a spinal dura mater is pushed away to remove intervertebral discs; after nerve roots are relaxed and free of compression, an internal fixation device with appropriate length and size is measured by a trial mold, and the internal fixation device is placed in the interspinous processes, with an outward extension of the rigid plate closely adhering to the vertebral laminae and covering the spinal canal, to reconstruct the spinal canal; and through the screw holes of the rigid plate on the side surfaces of the internal fixation device, the screws 3 are screwed into the spinous processes and the vertebral laminae, to fix the internal fixation device between the anterior and posterior spinous processes.

Practical application example 2: during posterior spinal surgery, a segment of the whole vertebral lamina spinous process is removed and decompressed, the yellow ligament is removed, the lateral recess nerve root canal is released, and the spinal dura mater is pushed away to remove intervertebral discs; after the nerve roots are relaxed and free of compression, spinous processes of the upper and lower segments and an interspinous process ligament are treated, and the spinous process bone graft surface is created; an internal fixation device with appropriate length and size is measured by the trial mold, and the internal fixation device is placed between the superior and inferior spinous processes, with an outward extension of the rigid plate closely adhering to the vertebral laminae and covering the spinal canal, to reconstruct the spinal canal; and through the screw holes of the rigid plate on the side surfaces of the internal fixation device, the screws 3 are screwed into the spinous processes and the vertebral laminae, to fix the internal fixation device between the anterior and posterior spinous processes.

Practical application example 3: during posterior spinal surgery, a supraspinous ligament and an interspinous ligament in the interspinous processes are removed, parts of the superior and inferior spinous processes and the vertebral lamina are treated, a spinous process bone graft surface is created; the interspinous processes is expanded, an internal fixation device with appropriate length and size is measured by the trial mold, and the internal fixation device is placed in the interspinous processes, with an outward extension of the rigid plate closely adhering to the vertebral laminae and covering the spinal canal, to reconstruct the spinal canal; and through the screw holes of the rigid plate on the side surfaces of the internal fixation device, the screws 3 are screwed into the spinous processes and the vertebral lamina, to fix the internal fixation device between the anterior and posterior spinous processes.

### Embodiment 2

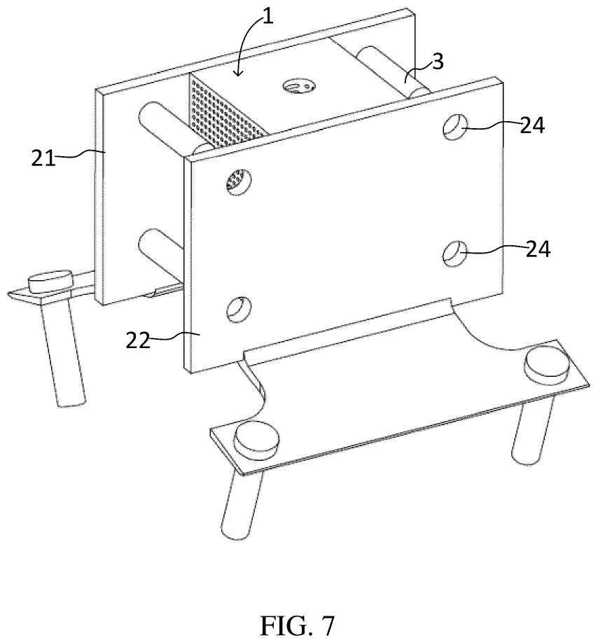

Referring to FIG. 7, an internal fixation device for fusion and reconstruction of spinous processes and vertebral laminae provided in the embodiment 2 is shown, including a bone fusion structure 1 and a rigid skeleton body 2. The rigid skeleton body 2 includes a first rigid plate 21, a second rigid plate 22 and an intermediate connecting plate 23. The intermediate connecting plate 23 is fixedly connected between the first rigid plate 21 and the second rigid plate 22, and the bone fusion structure 1 is located on the intermediate connecting plate 23. The first rigid plate 21 and the second rigid plate 22 are respectively located on the left side and the right side of the bone fusion structure 1.

A difference from the embodiment 1 is that the first rigid board 21 and the second rigid board 22 of the embodiment has the same structure, that is, both sides of the second rigid plate 22 are protruded from both front and back end surfaces of the bone fusion structure 1, and thus one or more screw holes 24 can be provided to connect with the anterior spinous process and the posterior spinous process. When using the screws 3 to fixedly connect to the bone tissue, the screws 3 can be screwed into the one or more screw holes 24 on the first rigid plate 21 and the second rigid plate 22, to achieve a fastened connection between the internal fixation device and the bone tissue.

### Embodiment 3

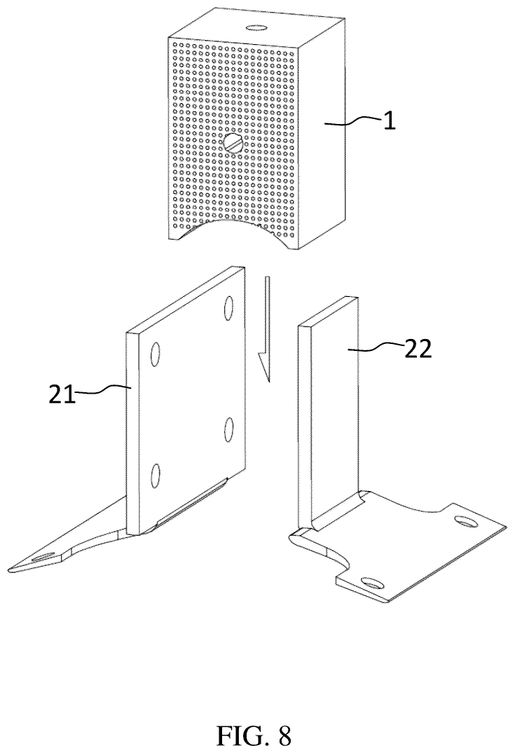

Referring to FIG. 8, an internal fixation device for fusion and reconstruction of spinous processes and vertebral laminae provided in the embodiment 3 is shown, including a bone fusion structure 1 and a rigid skeleton body 2. The rigid skeleton body 2 includes a first rigid plate 21 and a second rigid plate 22.

A difference from the embodiment 1 is that the rigid skeleton body 2 of the embodiment is a split structure, and consisting of two independent rigid plates (no intermediate connecting plate 23 of the embodiment 1). The first rigid plate 21 and the second rigid plate 22 can be fixed to the left and right sides of the bone fusion structure 1 by small screws respectively.

The internal fixation device for fusion and reconstruction of spinous processes and vertebral laminae provided by the present disclosure has been introduced in detail above, in the article, the principles and implementation modes of the present disclosure has been illustrated using specific examples, and the above description of the embodiments is only used to help understand the method and core idea of the present disclosure; at the same time, for those skilled in the art, according to the idea of the present disclosure, there will be changes in the specific implementation modes and application scope. In summary, the contents of the specification should not be construed as limiting the present application.

1. An internal fixation device for fusion and reconstruction of spinous processes and vertebral laminae, comprising a rigid skeleton body and a bone fusion structure,
the rigid skeleton body comprising a first rigid plate and a second rigid plate, located on a left side and a right side of the bone fusion structure respectively;
both the first rigid plate and the second rigid plate comprising:
a bone fusion structure fixed part, fixedly connected to the bone fusion structure; and
a vertebral lamina fixed part, wherein a top of the vertebral laminae fixed part is fixedly connected to a bottom of the bone fusion structure fixed part; and
the first rigid plate comprising:
an anterior spinous process fixed part and a posterior spinous process fixed part, fixedly connected to a front end and a back end of the bone fusion structure fixed part respectively, and protruded from a front end surface and a back end surface of the bone fusion structure respectively; and
wherein the anterior spinous process fixed part is provided with a first screw hole, and is connected to an anterior spinous process and an anterior vertebral lamina by a first screw; and the posterior spinous process fixed part is provided with a second screw hole, and is connected to a posterior spinous process and a posterior vertebral lamina by a second screw; and
the bone fusion structure located between the first rigid plate and a second rigid plate; wherein a first microporous structure is provided inside the bone fusion structure, and an orifice of the first microporous structure is located on a side surface of the bone fusion structure.

2. The internal fixation device of claim 1, wherein the vertebral lamina fixed part is a bending structure relative to the bone fusion structure fixed part, and the vertebral lamina fixed part is formed by the bottom of the bone fusion structure fixed part being inclined extended downward and into an outward direction.

3. The internal fixation device of claim 1, wherein a bottom shape of the bone fusion structure is matched with a top shape of the vertebral lamina, to make a bottom surface of the bone fusion structure fit a top surface of the vertebral lamina.

4. The internal fixation device of claim 1, wherein a storage channel configured to store bone fusion-promoting substances is located inside the bone fusion structure, and an inlet of the storage channel is located on a front side surface and a back side surface of the bone fusion structure.

5. The internal fixation device of claim 1, wherein contact positions of both the first rigid plate and the second rigid plate with human bones are provides with a second microporous structure.

6. The internal fixation device of claim 1, wherein a material of the bone fusion structure is titanium alloy, 3D printing materials, PEEK material, artificial bone or allogeneic bone.

7. The internal fixation device of claim 1, wherein a material of the rigid skeleton body is a metal material.

8. The internal fixation device of claim 1, wherein the first rigid plate and the second rigid plate are connected with the bone fusion structure by a third screw; or the bone fusion structure and the rigid skeleton body are an integral structure.

9. The internal fixation device of claim 2, wherein one end of the vertebral lamina fixed part connected to the bone fusion structure fixed part is a connecting end, the other end is a free end, and a width of the connecting end is smaller than a width of the free end.

10. The internal fixation device of claim 1, wherein the vertebral lamina fixed part is provided with a third screw hole, and is connected to the vertebral lamina by a fourth screw, or, the vertebral lamina fixed part is fixed to the vertebral lamina and an articular process by the fourth screw; or, the vertebral lamina fixed part is fixed to the articular process and a pedicle of vertebral arch by the fourth screw; and
wherein the rigid skeleton body comprises an intermediate connecting plate, the intermediate connecting plate is connected between a bottom of the first rigid plate and a bottom of the second rigid plate, and the bone fusion structure is located on the intermediate connecting plate.
